(19) United States
(12) Patent Application Publication
(10) Pub. No.: US 2021/0137219 A1
(43) Pub. Date: May 13, 2021

(54) APPARATUS FOR MOULDING INSOLES

(71) Applicant: Wintersteiger AG, Ried im Innkreis (AT)

(72) Inventors: Andreas Lachinger, Micheldorf (AT); Alexander Holzberger, Micheldorf (AT)

(73) Assignee: Wintersteiger AG, Ried im Innkreis (AT)

(21) Appl. No.: 16/965,397

(22) PCT Filed: Jan. 10, 2019
(86) PCT No.: PCT/AT2019/050003

§ 371 Date: Jul. 28, 2020

(30) Foreign Application Priority Data
Jan. 30, 2018 (AT) A50080/2018

(51) Int. Cl. A43D 1/02

(52) U.S. Cl. CPC A43D 1/022

(57) ABSTRACT

An apparatus for moulding shoe insoles onto a foot, having a moulding pad arranged on a support and forming a foot-accommodating region. The moulding pad may have a flexible membrane for supporting the insoles so that insoles can be moulded on without assistance and independently of a standing or sitting position of the user. The flexible membrane may be connected to a bearing surface of the support via a foot-bearing-point fixing means, and a filling region for a fluid for moulding the insole onto the foot between the membrane and the bearing surface may be provided.

### TECHNICAL FIELD

The system described herein relates to an apparatus for moulding shoe in-soles onto a foot, having a moulding pad arranged on a support and forming a foot-accommodating region.

### BACKGROUND OF THE INVENTION

There are known apparatuses for moulding shoe insoles (US 20160288440 A1), which show a pad arranged on a support and covered by a flexible protective cover. The pad has a layered structure, wherein the layer facing the wearer consists of a foam and the second layer, which is detachably arranged on top of it and forms a foot-accommodating region, consists of a gel-like material. During moulding, a pre-heated, thermoformable insole is placed in the foot-accommodating region and then a foot is placed on the inlay. In order for the insole to be moulded, the arch of the foot must at least partially penetrate the gel layer, which is only possible by applying greater forces, so that moulding can usually only occur by means of a weight force that can be applied when standing. In addition, the weight force cannot be applied evenly to the entire foot, so that the gel layer must be pressed against the foot, especially in the forefoot area, via an attached grip tab until the insole has cooled down in the moulded-on state and is thus dimensionally stable. Consequently, assistance is required during the moulding process and the unstable, flexible gel layer means that the user cannot be assured of a secure foothold.

### SUMMARY OF THE INVENTION

The system described herein thus may include an apparatus of the type described above designed in such a way that shoe insoles may be moulded on without assistance and independently of a standing or sitting position of the user.

In some embodiments of the system described herein, the moulding pad may have a flexible membrane for bearing the insole, which may be connected to a bearing surface of the wearer via a foot-bearing-point fixing means and a filling region for a fluid for moulding the insole to the foot may be provided between the membrane and the bearing surface. As a result of features according to embodiments of the system described herein, the foot of the user may remain in a rest position when the insole is moulded onto the foot, because the insole may be moulded onto the foot independently of the weight force applied by the user by filling the filling region with a fluid via the membrane.

The moulding pressure may be regulated by the filling quantity or the fluid pressure within the filling region, wherein advantageous moulding conditions may be obtained if the moulding pressure is selected in such a way that it allows a deformation of the insole on the one hand and on the other hand does not or only slightly exceeds the weight force of the foot of the user in sitting position. In order to nevertheless enable a reliable positioning of the insole and the foot in relation to each other and in particular to prevent the foot from slipping away, the flexible membrane may be connected according to an embodiment of the system described herein to a bearing surface of the wearer via a foot-bearing-point fixing means, so that the membrane cannot be lifted by the fluid in the area of the foot-bearing-point fixing means, so that a defined bearing area may be given between the insole and the foot. It may be advantageous that the foot-bearing-point fixing means is located in the heel section of the foot-accommodating region, because the user's heel allows a particularly easy positioning. However, it is also conceivable, for example, to position the foot-bearing-point fixing means in the section of the ball of the foot.

In order to facilitate the alignment of the insole to be moulded and the user's foot with respect to each other, an alignment aid with a flexible edge may be provided in the foot-accommodating region of the moulding pad. While the alignment aid already may be partially adapted to an ordinary foot shape in order to determine the position of the insole and the foot, the flexible edge area of the alignment aid may allow a reliable moulding depending on the actual shape and size of the user's foot.

In order to create particularly simple design conditions, the alignment aid may be connected to the bearing surface of the support via the foot-bearing-point fixing means. If the alignment aid is placed above the membrane, the filling region for the fluid, just like the membrane, only may have to be penetrated once, namely by the foot-bearing-point fixing means. In this case, the foot-bearing-point fixing means and the membrane should be fluid-tight.

In order to achieve a complete moulding of the insole to the shape of the user's foot even in the area of the preformed alignment aid, the alignment aid may be arranged within the filling region. This means that when filling the filling region with a fluid, the alignment aid may remain in its relative position to the bearing surface or to the wearer, while the membrane used for moulding the insole may lift off the alignment aid and hug the shape of the user's foot. When the fluid is emptied from the filling region, this feature also may allow the edge region of the alignment aid to be returned to its initial position, which may ensure reliable long-term operation, especially if an apparatus according to an embodiment of the system described herein is used frequently.

Depending on the size of the user's foot, the problem may arise that an undesirable moulding of the insole also may be formed in the toe area. In order to prevent this, the apparatus may have a slider to limit the stop of the membrane in the ball-side foot-accommodating region. Such a slider may provide an additional stable support point for the user's foot in the toe area, which may further facilitate the positioning of the foot.

An embodiment of the system described herein also relates to a method for moulding insoles with an apparatus, where the insole may be arranged on the membrane with the filling region largely empty and, after placing the foot on the insole, the filling region may be filled with a fluid, and that after moulding the insole, the filling region may be emptied to release the foot and the insole. When the filling region is largely emptied, the alignment aid thus may emerge through the membrane and facilitate the positioning of the insole and the foot. Particularly advantageous conditions may arise in connection with thermoformable insoles when the fluid used is cooled because this leads to a faster cooling of the preheated insole and thus to an earlier form stability. The fluid used may be air or water, for example, where the former may allow easy handling of the apparatus according to an embodiment of the system described herein.

### BRIEF DESCRIPTION OF THE DRAWINGS

The subject matter of embodiments of the system described herein is shown by way of example in the drawings, wherein:

FIG. 1 shows an apparatus in a schematic section with the filling region emptied, according to an embodiment of the system described herein;

FIG. 2 shows a representation corresponding to FIG. 1 with filled filling region and positioned foot of a user, according to an embodiment of the system described herein;

FIG. 3 shows an apparatus in a representation corresponding to FIG. 1, according to an embodiment of the system described herein; and

FIG. 4 shows a representation corresponding to FIG. 3 with filled filling region and positioned foot of a user, according to an embodiment of the system described herein.

### DESCRIPTION OF THE VARIOUS EMBODIMENTS

Illustrative embodiments of the system described herein will now be described in relation to the drawings. It should be appreciated that the system described herein is not limited to the following illustrative embodiments, as other embodiments, for example, variations of the following illustrative embodiments, are possible, and intended to fall within the scope of the invention.

An apparatus according to an embodiment of the system described herein comprises a moulding pad 3 arranged on a support 1 and forming a foot-accommodating region 2, which may have a flexible membrane 4 for supporting an insole 5. In addition, the membrane 4 may be connected to a bearing surface 7 of the support 1 via a foot-bearing-point fixing means 6. Between the membrane 4 and the bearing surface 7 of the support 1, a filling region 8 may be provided for a fluid for moulding the insole 5 to the foot 9 of a user. The filling region may be formed, for example, by the membrane forming a fillable bladder or by the membrane being fluid-tight to the support.

During a moulding process, the insole 5 may be first placed in the foot-accommodating region 2 of the membrane 4 and then the user's foot 9 may be placed on the insole 5. Subsequently, pressure may be applied to the membrane 4 by filling the initially empty filling region 8 with a fluid. As a result of the expansion of the membrane 4, the membrane may press the mouldable insole 5 against the foot 9. After the insole 5 has been adapted to the user's foot 9 in this way, the filling region 8 may be emptied again so that the foot 9 and the moulded-on insole 5 may be removed from the foot-accommodating region 2.

In order to simplify the alignment of the insole 5 and the foot 9 to each other, an alignment aid 10 may be provided in the foot-accommodating region 2 of the moulding pad 3 according to an embodiment of an apparatus of the system described herein. In order not to impede a moulding process despite the contour-giving design of the alignment aid 10, the alignment aid 10 may have a flexible edge area 11 so that the insole 5 may nestle against the user's foot 9 due to the application of pressure to the membrane 4.

In order to create simple design conditions, it may be provided according to an embodiment that the alignment aid 10 is connected to the bearing surface 7 of the support 1 via the foot-bearing-point fixing means 6. For example, the foot-bearing-point fixing means 6 may be screwed to the support 1.

In order to ensure that the membrane 4 may fully nestle against the user's foot 9 during moulding, the alignment aid 10 may be positioned within the filling region 8.

This may have the further advantage that, when emptying the filling region 8, the membrane 4 may nestle against the alignment aid 10 in such a way that the positioning of the insole 5 and foot 9 may be carried out even easier.

In order to avoid an undesired moulding of the insole 5 to the toe area, for example, with different shoe sizes, the apparatus also may have a slider 12 to limit the stop of the membrane 4 in the ball-side foot-accommodating region 2. In this case, the slide 12 may be moved in the longitudinal direction of the support prior to the moulding process in such a way that the length of the foot-accommodating region 2 may be adjusted and consequently the section of the insole 5 to be moulded may be determined.

If thermoformable insoles 5 are used, it may be provided, according to an embodiment of the system described herein, that the fluid flowing into the filling region 8 is cooled, so that the thermoformable insoles 5 may harden more quickly in a dimensionally stable state after they have been moulded to the foot 9.

In addition, according to an embodiment of an apparatus of the system described herein, a compliant buffer element 13, for example of a foam material, may be provided, which may be arranged between the alignment aid 10 and the bearing surface 7 of the support 1, in order to enable a more pleasant tactile perception of the user during the positioning process of the foot 9 when the filling region 8 is largely empty.

The system described herein is not restricted to the described embodiments. It may be varied within the scope of the claims, taking into account the knowledge of the relevant person skilled in the art. Other embodiments of the system described herein will be apparent to those skilled in the art from a consideration of the specification and/or an attempt to put into practice the system described herein. It is intended that the specification and examples be considered as illustrative only, with the true scope and spirit of the invention being indicated by the following claims.

1. An apparatus for moulding insoles onto a foot, having a moulding pad which is arranged on a support, forms a foot-accommodating region and has a flexible membrane for supporting the insole, wherein the membrane is connected to a bearing surface of the support via a foot-bearing-point fixing means, and wherein a filling region for a fluid for moulding the insole onto the foot is provided between the membrane and the bearing surface.

2. The apparatus according to claim 1, wherein an alignment aid is provided in the foot-accommodating region of the moulding pad, which has a flexible edge region.

3. The apparatus according to claim 2, wherein the alignment aid is connected to the bearing surface of the support via the foot-bearing-point fixing means.

4. The apparatus according to claim 2, wherein the alignment aid is arranged within the filling region.

5. The apparatus according to claim 1, wherein the apparatus has a slide for limiting the stop of the membrane in the foot-accommodating region on the ball side.

6. A method of moulding insoles with an apparatus including a moulding pad arranged on a support, wherein the moulding forms a foot-accommodating region and has a flexible membrane for supporting the insole, wherein the membrane is connected to a bearing surface of the support via a foot-bearing-point fixing means, and wherein a filling region for a fluid for moulding the insole onto the foot is provided between the membrane and the bearing surface, the method comprising:
, arranging the insole on the membrane with the filling region largely empty;
after placing the foot on the insole, filling the filling region with a fluid; and
after moulding the insole, emptying the filling region to release the foot and the insole.

7. The apparatus according to claim 3, wherein the alignment aid is arranged within the filling region.

8. The apparatus according to claim 2, wherein the apparatus has a slide for limiting the stop of the membrane in the foot-accommodating region on the ball side.

9. The apparatus according to claim 3, wherein the apparatus has a slide for limiting the stop of the membrane in the foot-accommodating region on the ball side.

10. The apparatus according to claim 4, wherein the apparatus has a slide for limiting the stop of the membrane in the foot-accommodating region on the ball side.

11. The method according to claim 6, wherein an alignment aid is provided in the foot-accommodating region of the moulding pad, which has a flexible edge region.

12. The method according to claim 6, wherein the alignment aid is connected to the bearing surface of the support via the foot-bearing-point fixing means.

13. The method according to claim 6, wherein the alignment aid is arranged within the filling region.

14. The method according to claim 6, wherein the apparatus has a slide for limiting the stop of the membrane in the foot-accommodating region on the ball side.

15. An apparatus for moulding insoles onto a foot, comprising:
a support;
a moulding pad arranged on the support, the moulding pad including a foot-accommodating region and a flexible membrane for supporting the insole, wherein the membrane is connected to a bearing surface of the support; and
a filling region for accommodating a fluid for moulding the insole onto the foot, wherein the filling region is located between the membrane and the bearing surface.

16. The apparatus according to claim 15, further comprising:
a foot-bearing-point fixing means connecting the membrane to a bearing surface of the support.

17. The apparatus according to claim 15, wherein the foot-accommodating region includes an alignment aid, wherein the foot-accommodating region has a flexible edge region.

18. The apparatus according to claim 17, further comprising:
a foot-bearing-point fixing means connecting the membrane to a bearing surface of the support, wherein the alignment aid is connected to the bearing surface of the support via the foot-bearing-point fixing means.

19. The apparatus according to claim 17, wherein the alignment aid is arranged within the filling region.

20. The apparatus according to claim 15, further comprising:
a slide limits a stop of the membrane in the foot-accommodating region on a ball side pf the foot.
